(19) United States
(12) Patent Application Publication
(10) Pub. No.: US 2025/0020633 A1
(43) Pub. Date: Jan. 16, 2025

(54) Discovering Active Components in Herbals by Manipulating Common Elements

(71) Applicant: Jianyi Zhang, Clarksville, MD (US)

(72) Inventor: Jianyi Zhang, Clarksville, MD (US)

(21) Appl. No.: 18/222,447

(22) Filed: Jul. 16, 2023

(51) Int. Cl. G01N 33/50

(52) U.S. Cl. CPC G01N 33/5038; G01N 2800/52

(57) ABSTRACT

Herbal or Traditional Chinese Medicine (TCM) is often mixed with multiple compounds. The effect may be from one or a few compounds, the reactants of several compounds, or the metabolism and reactants of the compound compounds in the body. Therefore, it is impossible to determine what bioactive compounds is/are by the traditional method.

In this method, herbals are used to treat patients and blood specimens are collected and subjected to chemical analysis. Common chemical elements are identified from patients who respond well to the herbals, and the common elements will be matched with elements from poorly responsive patients to find out which elements were missed. Adding missed elements and treating the poorly responsive patients, if the treated patients respond well, the common elements are bioactive ones.

### CROSS-REFERENCE TO RELATED APPLICATIONS

The present application relates and claims priority to U.S. provisional patent application No. 63/368,947, filed on Jul. 18, 2022.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### FIELD OF THE INVENTION

This invention relates to methods to identify bioactive compounds from herbals.

### Abbreviations

The terms “FDA” or “US FDA” refer to the Food and Drug Administration, a USA governmental agency that regulates food and drugs in the USA.

The term “LCMS” refers to liquid Chromatography and Mass Spectrometer

The term “CEs” refers to Common Elements.

The term “TCM” refers to Traditional Chinese Medicine.

CEs are comm. Elements that are u pounds present in all patients initially responsive to herbal treatments according to pre-set criteria.

### INTRODUCTION

Western medicine is the product of modern chemical synthesis. These compounds are synthesized in the laboratory. A few are natural substances, and most of them are new molecules.

Current methods of developing new medicine cost a few billion, and only one new compound is tested. Testing two or more new compounds in the same test would cost a few times more, and no pharmaceutical company would take such a risk.

Herbal or Traditional Chinese Medicine (TCM) is often mixed with multiple compounds. The effect may be from one or a few compounds, the reactants of several compounds, or the metabolism and reactants of the compound compounds in the body. Therefore, it is impossible to determine what bioactive compounds is/are by the traditional method.

### DESCRIPTION

Two conditions must be met for therapeutic effects: first, all bioactive compounds must be present and detectable in the blood, and second, there must be a complete reaction chain in cells or tissues in the body.

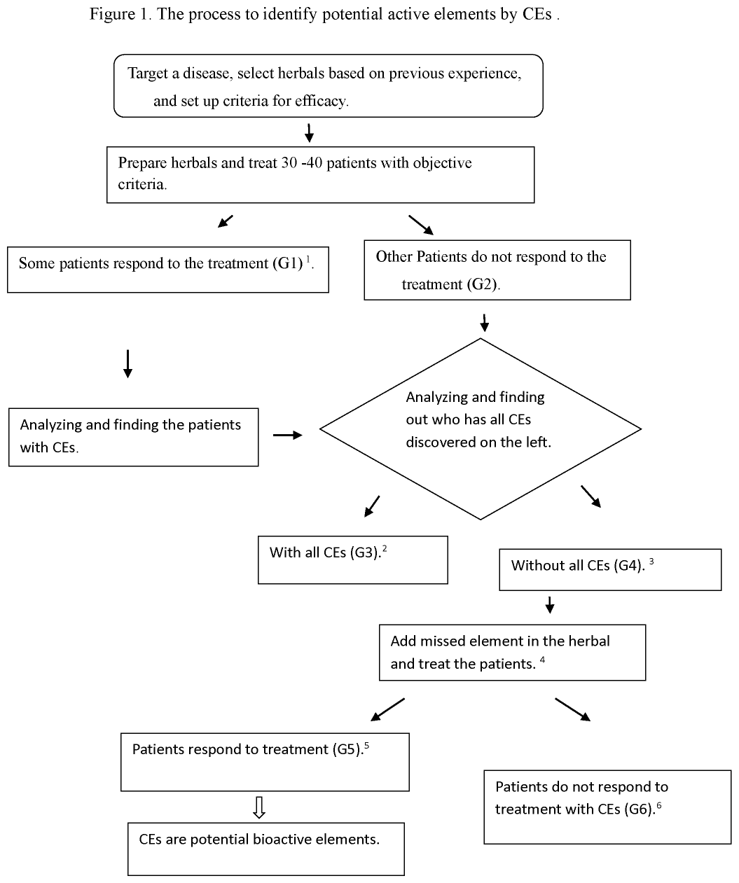

Take a concoction as an example; bioactive components can be identified through the following steps (all numbers below are assumed):
1. First, set up objective criteria for effects, such as white blood cell count for infection, blood sugar for diabetes, and X-ray for lung cancer.
2. Treat 30-40 patients with herbal or mixed herbs and draw blood before and after treatment.
3. Twenty of these patients (assumed) have responded by pre-set criteria (group 1); the rest do not (group 2).
4. To analyze the blood samples of all patients with the chemical instruments LCMS, there will be many compounds of elements present in the patients.
5. To determine what elements are present in all G1 patients, assumed three peaks (C1, C2, C3) are found in all patients in group 1, they are considered as common elements or CEs.
6. To analyze and see if G2 patients to have all CEs.
7. After analysis, a few patients have all CEs in their blood (G3), and the others have only some or none of (G4).
8. To treat G4 patients with CEs if they demonstrate a desired response (G5). If yes, these CEs are identified as bioactive ones.
9. The G6 patients without effect could be due to the lack of a complete reaction chain, even if they have all CEs (see FIG. 1).

### Embodiment: Treating Patients with Bacterial Infection

Tulsi (Ocimum teniflorum) has main components including eugenol (1-hydroxy-2-methoxy-4-allylbenzene); Cinnamon (Cinnamomu m verum) has main components including eugenol, cinnamaldehyde, beta-caryophyllene, linalool, and methyl chavicol. Clove (Syzygium aromaticum) main components including phenylpropanoides such as carvacrol, thymol, eugenol and cinnamaldehyde.

The three herbals were mixed in ratio) 1:2,2.5, blended and made into 350 mg capsule.

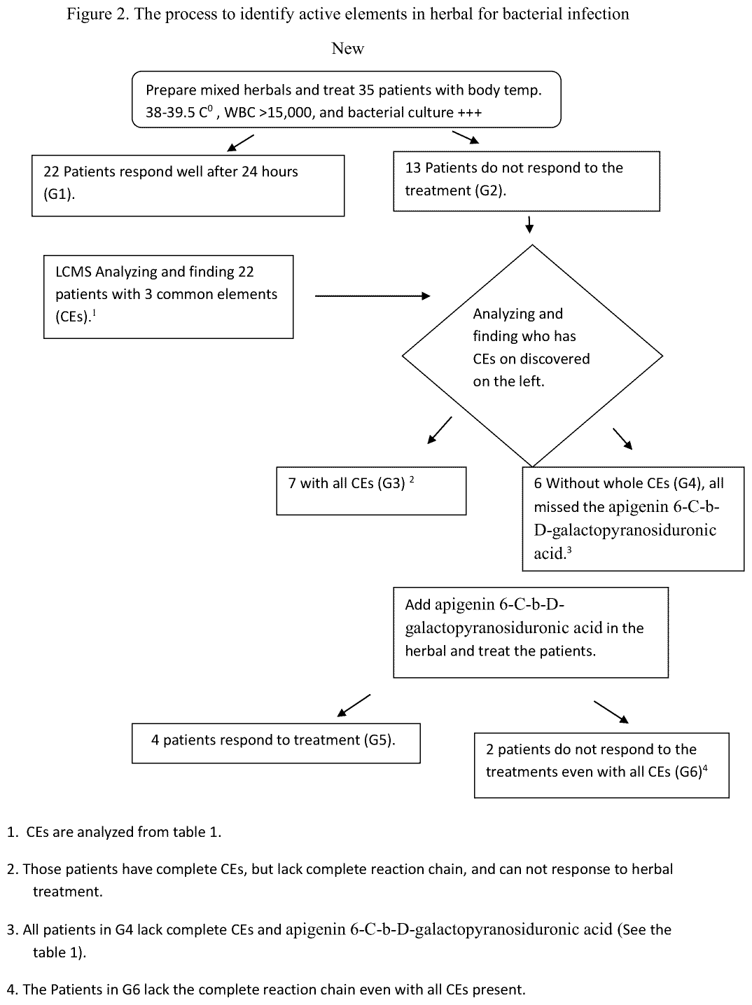

Major steps are as follows:
1. Enroll 35 patients with 38-39.5° C., WBC>15,000 and bacterial culture ++
2. Treat the patients with the mixed herbals, 350 mg two times a day, and draw blood after 24 hours, check body temperature, do complete blood count, culture blood.
3 Twenty-two of them respond well with the treatment (group 1), and the thirteen do not respond well with treatment (G2).
4. To analyze the blood samples of all 35 patients with LCMS by the standard method, there will be over 35 peaks
5. To determine what peaks are present in all group 1 patients by the method in the table 1, three peaks (C1-C3) are found in all group 1 patients, considered as common elements or CEs (See the table 1).
6. To analyze the peaks of group 2 patients to see if they have complete CEs.
7. Seven patients have all CEs (the group 3), and the rest of the six patients do not have all CEs (the group 4).
8. Send application for medical ethnic board for treating patients with CEs, if approved recruit patients for further studies.
9. To treat around 4 patients with CEs, six of them demonstrate the desired response (group 5), these C s are identified as bioactive one, two patients do not (the group 6) The above process is demonstrated in the FIG. 2.
10. The three CEs are cinnamaldehyde (from Clove) 1-hydroxy-2-methoxy-4-allylbenzene (from), third one is apigenin 6-C-b-D-galactopyranosiduronic acid that is not present in the three herbals, is a reactant in human body.
11. All of these elements are present in the patients, and there should be little issue with their safety. Currently, there are no regulations regarding how to apply identified compounds in humans. Even if some experiments need to be done before they can be used in humans, the processes will be much simpler.
12. The initial phase is to identify CEs in the patients, which are based on observation in the clinic, not a clinical trial. There might not be necessary for approval by the medical ethnic board
13. Potential bioactive elements may be available on the market; if not, they can be enriched by purification or synthesizing them with human effort. Since the active ingredients can be directly made into tablets, their quality and quantity can be guaranteed.
14. In the above case, the missed element is apigenin 6-C-b-D-galactopyranosiduronic acid that is not present in any of three herbs, and is a reactant in the body.
15. In the process, the time is considerably shortened, and the number of patients is significantly reduced. Under the objective indicators, the changes before and after treatment are the compared, and there is no need for other control groups. Cost can be 1/1,000, 1/10,000, or less than traditional methods.
16. The identified active ingredient may be one or several elements in the herb, their reactants, or their metabolites in the body. Bioactive elements can act on multiple sites, preventing and treating diseases from numerous perspectives and achieving results that traditional methods cannot reach.
17. LCMS is a standard methodology in pharmaceutical study, which is not a part of the invention.
18. This method is to identify bioactive elements from patients who have been proven safe and effectively treated with herbals, which makes many steps of traditional methods unnecessary. It is just the opposite of conventional techniques, so it is also called reverse analysis.
19. Depending on the presence of reaction chain and synthetic ability, patients can be classified into 4 groups

| | Synthase ability | Reaction chain | Group No. | Complete CE | Desired response |
|---|---|---|---|---|---|
| 1 | Present | Present | 1 | Yes | Yes |
| 2 | Present | No complete | 3 | Yes | No |
| 3 | No present | Present | 5 | No | No |
| 4 | No | No | 6 | NO | No |

### REFERENCES

Shami M. A. Fadl F. A. Siry A. R. et al. (1985) Antifungal property of garlic clove juice compared with fungicidal treatment against Fusarium with watermelon Egyptian Journal of Phytopathology, 17:
Cosentino S. Tuberoso C. I. G., Pisano B., et al (1999) In-vitro antimicrobial activity and chemical composition of Sardinian thymus essential oils. Letters in Applied Microbiology, 29:130-135
Mothana R. A. Lindequist U. (2005) Antimicrobial activity of some medicinal plants of the island Sogotra, Journal of Ethno pharmacology, 96 (1-2): 177-181.

TABLE 1
Identifying Common Elements in Patients Respond to Herbal Treatment
New

| | P1/2 | P3/6 | P4/9 | P5/11 | P6/10 | P7/8 | P12/15 | P16/18 | P19/22 | P13/21 | P14/17 |
|---|---|---|---|---|---|---|---|---|---|---|---|
| C1 | xx | | xx | xx | xx | | xx | | xx | xx | xx |
| C2 | | xx | xx | | xx | | xx | xx | xx | xx | |
| C3 | xx | xx | xx | xx | xx | xx | xx | xx | xx | xx | xx |
| C4 | xx | xx | xx | | | xx | xx | | | xx | |
| C5 | xx | xx | xx | xx | xx | xx | xx | xx | xx | xx | xx |
| C6 | xx | | xx | xx | xx | xx | | xx | xx | xx | |
| C7 | | xx | | xx | | | xx | xx | | xx | xx |
| C8 | xx | | xx | xx | xx | | xx | xx | | xx | xx |
| C9 | xx | xx | xx | xx | xx | xx | xx | xx | xx | xx | xx |
| C10 | xx | | | xx | xx | | xx | xx | xx | | |
| C11 | xx | | xx | xx | | xx | xx | | xx | xx | xx |

1. xx: compound present, blank: compound not present.
2. There are 11 elements (C1-C11), and 22 patients (P1-P22) responded to the herbal treatment.
3. Only C3, C5 and C9 are present in all patients, they are common elements.

1. A method of identifying potential bioactive compounds of herbals, including the steps of: setting up criteria for effectiveness with herbal treatments; selecting herbals; treating patients and separating patients who respond effectively and non-effectively, preparing and analyzing patients' blood, identifying common elements (CES) in from patients who meet pre-set criteria after treatment; identifying all elements in non-effective patients, selecting patients from non-effective ones if the patients lack whole or partial CEs, treating selected patients with CEs with adding the missed CEs either by purified or synthesized CEs, confirming CEs are active ones if treated patients who meet pre-set criteria to the treatment.

2. The method of claim 1 comparing and identifying CEs in patients who meet pre-set criteria to herbal treatment by results of chemical analysis.

3. The method of claim 1: From patients who do not meet pre-set criteria, select patients who missed whole or partial CEs based on chemical analysis results.

4. The method of claim 1: treating patients in claim 3 by administering CEs by either market-available or synthesized CEs, confirming that CEs are bioactive components if the patients become ones who meet pre-set criteria after the treatment.
